(19) United States
(12) Patent Application Publication
(10) Pub. No.: US 2026/0325162 A1
(43) Pub. Date: Oct. 22, 2026

(54) A Rotational Atherectomy Device

(71) Applicant: CLEARSTREAM TECHNOLOGIES LIMITED, Enniscorthy, County Wexford (IE)

(72) Inventors: Artur Spodobalski, Enniscorthy, County Wexford (IE); Allan Ronan, Enniscorthy, County Wexford (IE); Eoin Connaughton, Gorey, County Wexford (IE)

(21) Appl. No.: 19/104,766

(22) PCT Filed: Sep. 13, 2022
(86) PCT No.: PCT/EP2022/075391

§ 371 Date: Feb. 19, 2025

(51) Int. Cl. A61B 17/3207; A61B 17/00; A61B 17/32

(52) U.S. Cl. CPC A61B 17/320725; A61B 17/00234; A61B 2017/00305; A61B 2017/00398; A61B 2017/00477; A61B 2017/00778; A61B 2017/320004

(57) ABSTRACT

A rotational atherectomy device for cutting or abrading tissue. The atherectomy device comprises a rotatable inner shaft having a longitudinal axis, a plurality of blades connected to the distal end of the rotatable inner shaft through a spring mechanism which biases the plurality of blades radially outward, and an outer shaft disposed over the rotatable inner shaft. The rotatable inner shaft is longitudinally moveable with respect to the outer shaft, and distal movement of the inner shaft with respect to the outer shaft moves the plurality of blades from a radially contracted configuration to a radially expanded configuration.

### TECHNICAL FIELD

The present disclosure relates to a rotational atherectomy device for cutting or abrading tissue, and a method of cutting or abrading tissue using a rotatable atherectomy device.

### BACKGROUND

Atherosclerosis is a condition where calcified plaque deposits in the walls of a blood vessel lead to narrowing of the blood vessel and increased blood pressure. In severe cases, this can result in peripheral artery disease, coronary artery disease, tissue death or stroke.

Atherosclerosis can be treated in a number of ways such as by angioplasty, where a balloon is used to force the expansion of the blood vessel to allow improved blood flow, a vascular bypass, where a surgical procedure is performed to redirect the blood flow to circumvent the diseased area of the blood vessel, or atherectomy.

Atherectomy is a non-surgical procedure to open blocked blood vessels using a device at the distal end of a catheter to cut or grind atherosclerotic plaque from the walls of the blood vessel. Atherectomy devices can generally be classified into four different types: orbital, rotational, laser and directional.

A rotational atherectomy device uses a rotating abrasive head to cut or grind the atherosclerotic plaque from the walls of the blood vessel. A number of existing rotational atherectomy devices incorporate an abrasive head of fixed diameter. In order to treat larger diameter vessels, the abrasive head is offset towards one side of the vessel wall and the lesion is treated with multiple axial passes. This makes the treatment procedure longer and more complex.

There is hence a need in the art for a new type of rotational atherectomy device,, which allows controlled and precise expansion of the abrasive head and therefore reduce the overall treatment time as larger vessels can be treated in fewer axial passes of the abrasive head.

### SUMMARY

In a first aspect of the present invention, there is provided a rotational atherectomy device for cutting or abrading tissue. The atherectomy device comprises a rotatable inner shaft having a longitudinal axis; a plurality of blades connected to the distal end of the rotatable inner shaft through a spring mechanism which biases the plurality of blades radially outward; and an outer shaft disposed over the rotatable inner shaft. The rotatable inner shaft is longitudinally moveable with respect to the outer shaft. Distal movement of the inner shaft with respect to the outer shaft moves the plurality of blades from a radially contracted configuration to a radially expanded configuration.

In some embodiments, this may result in an atherectomy device which allows controlled and precise expansion of the plurality of blades to allow vessels of different sizes to be treated.

The spring mechanism may comprise a leaf spring or a torsion spring.

In some embodiments, this may provide for a robust way to bias the plurality of blades outward.

The outer shaft may be rotatable.

In some embodiments, this may reduce friction between outer shaft and the rotatable inner shaft, thereby making the rotational atherectomy device more robust.

The rotatable outer shaft may be configured to rotate together with the rotatable inner shaft.

The rotatable outer shaft may be configured to rotate at the same speed as the rotatable inner shaft. The rotatable outer shaft may be configured to rotate in the same direction as the rotatable inner shaft.

In some embodiments, this may reduce friction between the outer shaft and the rotatable inner shaft, thereby making the rotational atherectomy device more robust.

The plurality of blades may comprise 2 to 10 blades. The plurality of blades may comprise 2 to 6 blades.

In some embodiments, this may result in more effective removal of plaque by the rotational atherectomy device.

At least one of the plurality of blades may comprise a cutting edge. Each one of the plurality of blades may comprise a cutting edge.

In some embodiments, this may result in more effective removal of plaque from the vessel wall by cutting the plaque.

The cutting edge may be positioned radially inwardly offset from a radial outermost point of the blade.

In some embodiments, this may minimise damage to the vessel wall.

The cutting edge may be disposed at a circumferential edge of the blade.

In some embodiments, this may result in more effective cutting of plaque. In some embodiments, this may also minimise damage to the vessel wall.

At least one of the plurality of blades may have a claw-shaped axial cross section. Each of the plurality of blades may have a claw-shaped axial cross section.

Throughout this disclosure, the term “axial cross section” will be used to refer to a cross section which is perpendicular to the longitudinal axis.

Throughout this disclosure, the term “claw-shaped” will be used to refer to a shape which is curved and tapered towards a point or edge.

In some embodiments, this may minimise damage to the vessel wall while providing for effective cutting of plaque from the vessel wall.

At least one of the plurality of blades may comprise an abrasive surface. Each one of the plurality of blades may comprise an abrasive surface.

In some embodiments, this may result in more effective of removal of plaque from the vessel wall by grinding the plaque.

The abrasive surface may be disposed on a radial outer surface of the blade.

The radial outer surface of at least one of the plurality of blades may have a curved convex shape. The radial outer surface of each one of the plurality of blades may have a curved convex shape.

In some embodiments, this may minimise damage to the vessel wall when the blades are rotating.

The plurality of blades may be substantially straight along a longitudinal direction.

In some embodiments, this may allow the amount of expansion of the plurality of blades to be more accurately controlled by longitudinal movement of the inner rotatable shaft relative to the outer shaft.

The outer shaft may be in contact with at least one of the plurality of blades. The outer shaft may be in contact with each of the plurality of blades.

In some embodiments, this may allow the expansion of the plurality of blades to be more accurately controlled by longitudinal movement of the inner rotatable shaft relative to the outer shaft.

The outer shaft may cover the plurality of blades in the radially contracted configuration.

In some embodiments, this may minimise vessel damage while advancing the plurality of blades to a treatment site.

The amount of distal movement of the rotatable inner shaft relative to the outer shaft may determine the amount of radial expansion of the blades.

In some embodiments, this may provide a more accurate way to control the amount of expansion of the plurality of blades.

The rotatable inner shaft may be a flexible shaft.

In some embodiments, this may allow the atherectomy device to be more easily advanced through tortuous vessels.

The outer shaft may be a flexible shaft.

In some embodiments, this may allow the atherectomy device to be more easily advanced through tortuous vessels.

The rotational atherectomy may further comprise a control unit connected at the proximal end of the rotatable inner shaft and the outer shaft.

The control unit may comprise a rotational motor for providing rotation to the rotatable inner shaft.

The control unit may comprise a rotation control mechanism for controlling the speed of rotation of the rotational motor.

In some embodiments, this may provide precise control over the speed of rotation of the plurality of blades. This may result in more effective plaque removal.

The control unit may comprise an expansion control mechanism for moving the rotatable inner shaft longitudinally with respect to the rotatable outer shaft to thereby move the plurality of blades between the radially contracted configuration and the radially expanded configuration.

In some embodiments, this may provide precise control over the amount of expansion of the plurality of blades. This may allow vessels of different sizes to be treated more effectively and more quickly.

The expansion control mechanism may comprise a rack and pinion.

The rotatable inner shaft may further comprises a guidewire lumen for accommodating a guidewire.

In some embodiments, this may allow a guidewire to be used to track the atherectomy device to a treatment site.

In a second aspect of the present disclosure, there is provided a method of cutting or abrading tissue using a rotatable atherectomy device comprising a rotatable inner shaft, a plurality of blades connected to the distal end of the rotatable inner shaft through a spring mechanism which biases the plurality of blades radially outward, and an outer shaft disposed over the rotatable inner shaft. The method comprising introducing the atherectomy device into a blood vessel; advancing the atherectomy device to a position in the blood vessel where tissue is to be cut or abraded; radially expanding the blades to the diameter of the vessel by distally advancing the inner shaft; rotating the inner shaft to cut or abrade the tissue.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a rotational atherectomy device according to one or more embodiments of the present disclosure;

FIG. 2A shows a cross-sectional side view of a distal end of the rotational atherectomy device of FIG. 1, according to one or more embodiments of the present disclosure;

FIG. 2B shows an axial cross section of a distal end of the rotational atherectomy device of FIG. 1, according to one or more embodiments of the present disclosure;

FIG. 3A shows a distal end of the rotational atherectomy device of FIG. 1 positioned in an artery, according to one or more embodiments of the present disclosure;

FIG. 3B shows a distal end of the rotational atherectomy device of FIG. 1 positioned in an artery with the plurality of blades expanding, according to one or more embodiments of the present disclosure;

FIG. 3C shows a distal end of the rotational atherectomy device of FIG. 1 positioned in an artery with the plurality of blades expanded, according to one or more embodiments of the present disclosure.

### DETAILED DESCRIPTION

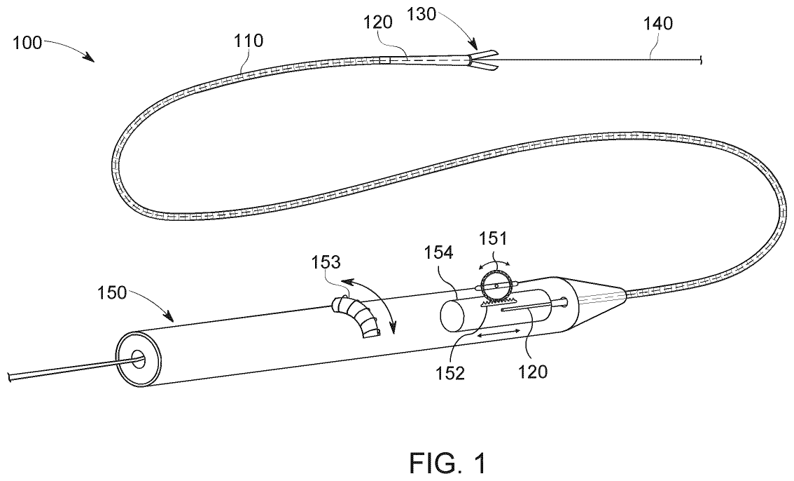

FIG. 1 shows a rotational atherectomy device 100 for cutting or abrading tissue and, specifically, for cutting or abrading atherosclerotic plaque from the walls of a blood vessel. The rotational atherectomy device 100 has an outer shaft 110 and a rotatable inner shaft 120 which is positioned within the outer shaft 110. A plurality of blades 130 are connected to a distal end of the rotatable inner shaft 120. A control unit 150 may be connected at a proximal end of the rotatable inner shaft 120 and the outer shaft 110. The outer shaft 110 may be rotatable and may specifically be configured to rotate with the rotatable inner shaft 120 such that the outer shaft 110 and rotatable inner shaft 120 rotate in the same direction and at the same speed. For example, the outer shaft 110 may be connected to the inner shaft 120 such that it rotates together with the inner shaft 120 but allows relative movement in a longitudinal direction. Furthermore, the rotational atherectomy device 100 may comprise a guidewire 140 positioned within a guidewire lumen of the inner rotatable shaft 120. The guidewire 140 may also be moveable relative to the rotatable inner shaft 120.

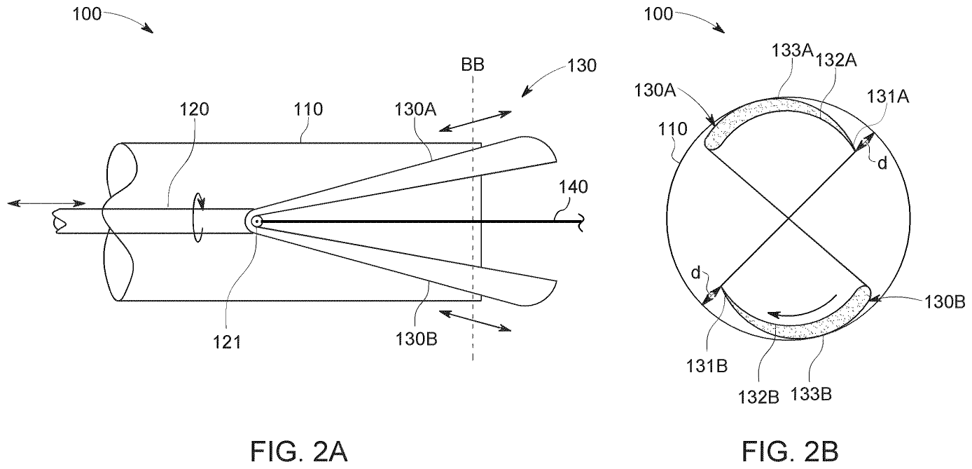

The plurality of blades 130 are connected to the distal end of the rotatable inner shaft 110 with a spring mechanism (see FIG. 2), such that the plurality of blades 130 are biased radially outwards. The plurality of blades 130 rotate together with the rotatable inner shaft 120 and can cut or abrade plaque from the inner walls of a blood vessel. The rotational atherectomy device 100 has two blades, however, it may comprise in the range of 2 to 10 blades, such as 2 to 6 blades. The rotatable inner shaft 120 is longitudinally moveable relative to the outer shaft 110. When the outer shaft 110 completely covers the plurality of blades 130, the plurality of blades 130 are in a radially contracted configuration, where the radial extent of the plurality of blades 130 is smaller than in a radially expanded configuration. When the rotatable inner shaft 120 is moved distally relative to the outer shaft 120, the plurality of blades 130 move distally beyond the distal end of the outer shaft 120. Due to the spring mechanism biasing the plurality of blades 130 radially outwards, the plurality of blades 130 will move from the radially contracted configuration to the radially expanded configuration, where the radial extent of the plurality of blades 130 is greater than in the radially contracted configuration. The amount of expansion of the plurality of blades 130 can be controlled by the amount of distal displacement of the rotatable inner shaft 120 relative to the outer shaft 110.

The control unit 150 may be in the form of a handle and may comprise a motor 154 connected to the rotatable inner shaft 120 for applying rotational motion to the rotatable inner shaft 120 and thereby the plurality of blades 130. The motor 154 may also be connected to the outer shaft 110. The control unit 120 may further comprise a rotation control mechanism which is connected to the motor 154 and allows the rotational speed of the motor and thereby the rotational speed of the rotatable inner shaft 120 to be controlled. As shown in FIG. 1, the rotation control mechanism may be in the form of a wheel 153 which can be rotated to increase or decrease the motor speed. The control handle 150 may also comprise an expansion control mechanism for moving the rotatable inner shaft 120 longitudinally relative to the outer shaft 110 and thereby causes the plurality of blades 130 to be moved between the radially contracted configuration and the radially expanded configuration. By moving the rotatable inner shaft 120 longitudinally relative to the outer shaft 110, the amount of expansion of the plurality of blades 130 can be controlled. The expansion control mechanism can, for example, comprise pinion 151 and a rack 152, where the pinion 151 can be rotated to move the rotatable inner shaft 120 proximally or distally relative to the outer shaft 110. The rack 152 may be connected to the motor 154 to move the motor 154 and rotatable inner shaft 120 longitudinally.

FIG. 2A shows a cross sectional side view of the distal end of the rotational atherectomy device 100. The plurality of blades 130 comprises a first blade 130a and a second blade 130b, but may comprise more than the two blades shown in FIG. 2A. Each of the plurality of blades 130 is connected to the rotatable inner shaft 120 via a spring mechanism 121 which may, for example, comprise a leaf spring or a torsion spring. Each of the first blade 130A and the second blade 130B are connected to the leaf spring 121 which biases the first blade 130A and the second blade 130B radially outwards. The leaf spring 121 biases the plurality of blades 130 radially outwards into contact with the outer shaft 110, which restricts the radial extent of the plurality of blades 130. When the rotating inner shaft 110 and the plurality of blades are advanced distally, the plurality of blades 130 will expand by moving distally and radially outwardly. The further distally the rotating inner shaft 120 and the plurality of blades 130 are advanced, the greater the radial extent of the plurality of blades 130.

Each of the plurality of blades 130 is substantially straight along a longitudinal axis. The straight shape of the plurality of blades 130 will help the plurality of blades 130 to expand uniformly when the rotatable inner shaft 120 is moved distally. The amount of expansion of the plurality of blades 130 can therefore be more accurately and precisely controlled via distal movement of the rotatable inner shaft 120.

The plurality of blades 130 may be made from a number of suitable materials such as, but not limited to, a cobalt base alloy, oxidised zirconium, tungsten, titanium or steel.

The guidewire 140 may be positioned within a lumen of the rotatable inner shaft 120 which may have an opening at the distal end of the rotatable inner shaft 120 such that the guidewire 140 can move longitudinally relative to the rotatable inner shaft 120 and move distally past the rotatable inner shaft 120.

FIG. 2B shows an axial cross section of the rotational atherectomy device 100 along line BB. Each of the plurality of blades 130A, 130B may have a claw-shaped cross section. For example, the first blade 130B may have a curved radial outer surface 133A and a curved radial inner surface 132A which taper towards and meet at a circumferential edge 131A. Similarly, the second blade 130B may have a curved radial outer surface 133B and a curved radial inner surface 132B which taper towards and meet at a circumferential edge 131B. The radial outer surface 133A, 133B may have a convex curved shape so as to be atraumatic to the vessel wall when the plurality of blades 130 are rotated.

The circumferential edge 131A, 131B of each of the blades 130A, 130B may be a sharp cutting edge for cutting the plaque from the vessel walls when the plurality of blades 130 are rotating. The radial outer surface 133A, 133B of each of the blades 130A, 130B may have an abrasive surface which is rough for grinding away plaque from the walls of the vessel through friction. The cutting edges which are disposed at the circumferential edges 131A, 131B are positioned radially inwardly offset by a distance d from a radial outermost point of the blade 130A, 130B. This reduces contact of the cutting edges 131A, 131B with the vessel wall when the blades 130A, 130B are rotating and thereby minimises damage to the vessel walls.

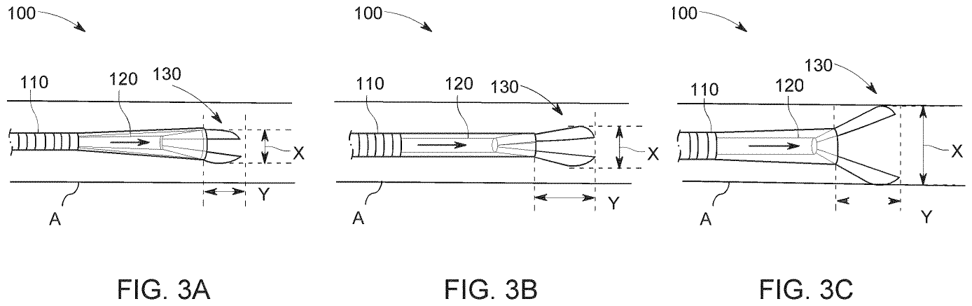

FIGS. 3A to 3C illustrate a method of expanding the plurality of blades 130 of the rotational atherectomy device 100 inside an artery A.

The amount of radial expansion x of the plurality of blades 130 can be controlled by controlling the amount distal displacement y of the rotatable inner shaft 120 relative to the outer shaft 110. FIG. 3A shows a configuration where the rotatable inner shaft 120 has been advanced by a small amount of distal displacement y resulting in a small amount of radial expansion x of the plurality of blades 130. FIG. 3B shows a configuration where the rotatable inner shaft 120 has been advanced by a greater amount of distal displacement y relative to the outer shaft 110 which results in a greater amount of radial expansion x of the plurality of blades 130. FIG. 3C shows an even greater amount of radial expansion x of the plurality of blades 130 resulting from an even greater amount of distal displacement y.

A method of using the rotational atherectomy device 100 in order to remove calcified plaque from a vessel wall of a patient will also be explained with reference to FIGS. 3A to 3C.

First a suitable access site may be identified. A guidewire 140 (see FIG. 1) may then introduced into the vessel, for example an artery A, through the access site and advanced to the treatment site. The treatment site may be, for example, a heavily calcified section of artery A where the vessel is significantly narrowed or even fully blocked such that blood flow through the artery A is significantly reduced. The rotatable inner shaft 120 together with the plurality of blades 130 and the outer shaft 110 are then introduced into the blood vessel over the guidewire 140 and through the access site. The outer shaft 130 is disposed over the plurality of blades 130 so that the plurality of blades 130 are in the radially contracted configuration. The rotatable atherectomy device 100 may be disposed within a sheath (not shown) for introduction into the vessel and advancement to the treatment site.

The atherectomy device 100 may then be advanced over the guidewire 140 until the plurality of blades 130 are positioned at the treatment site within artery A. As shown in FIG. 3A, once the plurality of blades are positioned at the treatment site, the rotatable inner shaft 120 can be advanced distally relative to the outer shaft 110, via the expansion control mechanism, for example, by rotating pinion 151. This will result in the plurality of blades 130 expanding from the radially contracted configuration to the radially expanded configuration. The amount of radial expansion x of the plurality of blades 130 can be controlled by controlling the amount of distal displacement y of the rotatable inner shaft 120 relative to the outer shaft 110, as illustrated in FIGS. 3A-3C and explained above. The physician can therefore accurately adjust the amount of radial expansion x of the plurality of blades 130 to match the diameter of the vessel to be treated.

Once the plurality of blades 130 have been moved from the radially contracted configuration to the radially expanded configuration and the extent of radial expansion x of the plurality of blades 130 has been matched to the diameter of the vessel to be treated, as shown in FIG. 3C, the physician can turn on the rotational motor using the rotational control mechanism, for example, by rotating wheel 153 (see FIG. 1) which will cause the rotatable inner shaft 120 and plurality of blades 130 rotate. The rotation will cause the plurality of blades 130 to cut or scrape calcified plaque from the inside of the vessel wall. The speed of rotation of the plurality of blades 130 can be controlled by adjusting the amount of rotation of the wheel 153 of the rotation control mechanism.

This atherectomy device 100 therefore allows for controlled and precise expansion of the plurality of blades 130 and therefore reduce the overall treatment time as larger vessels can be treated in fewer axial passes.

Various modifications will be apparent to those skilled in the art.

The spring mechanism 121 is not limited to a leaf spring, but may comprise any other suitable mechanism for biasing the plurality of blades 130 radially outwards. For example, the spring mechanism 121 may comprise a torsion spring.

The outer shaft 110 may not rotate together with the rotatable inner shaft 120. The outer shaft 100 may not rotate at the same speed or in the same direction as the rotatable inner shaft. The outer shaft 110 may not be rotatable at all.

The rotational atherectomy device 100 is not limited to having 2 to 10 blades but may comprise any suitable number of blades.

The plurality of blades 130 may not comprise a cutting edge.

At least one of the plurality of blades 130 may comprise more than one cutting edge. For example, each blade may comprise two circumferential edges with a cutting blade at each end of the blade.

The cutting edge is not limited to being disposed at the circumferential edge of the blade. For example, the cutting edge may be disposed at the radial inner surface of the blade or the radial outer surface of the blade.

The plurality of blades 130 may not comprise an abrasive surface.

At least one of the plurality of blades 130 may comprise more than one abrasive surface.

The abrasive surface is not limited to being disposed on the radial outer surface 133A, 133B of the blade 130A, 130B, but may be disposed, for example, on the radial inner surface 132A, 132B of the blade.

The plurality of blades 130 may comprise a mix of blades with cutting edges and blades with abrasive surfaces.

The plurality of blades 130 are not limited to having a claw-shaped axial cross section but may have any other suitable axial cross section. For example, the plurality of blades 130 may have a crescent-shaped axial cross section.

The plurality of blades 130 are not limited to being substantially straight along a longitudinal axis. For example, the plurality of blades may be curved along a longitudinal axis.

The atherectomy device 100 may not comprise a guidewire 140.

The rotatable inner shaft 120 may not comprise a guidewire lumen.

The atherectomy device 100 may not comprise a control unit 150.

The control unit 150 may not comprise an expansion control mechanism or a rotation control mechanism.

The rotation control mechanism is not limited to a wheel 151 but may take another suitable form such as a slider or a rotatable knob, for example.

The expansion control mechanism is not limited to a rack 152 and pinion 151 but may take another suitable form such as a slider, for example.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A rotational atherectomy device for cutting or abrading tissue, the atherectomy device comprising:
a rotatable inner shaft having a longitudinal axis;
a plurality of blades connected to the distal end of the rotatable inner shaft through a spring mechanism which biases the plurality of blades radially outward; and
an outer shaft disposed over the rotatable inner shaft, wherein the rotatable inner shaft is longitudinally moveable with respect to the outer shaft, and wherein distal movement of the inner shaft with respect to the outer shaft moves the plurality of blades from a radially contracted configuration to a radially expanded configuration.

2. The rotational atherectomy device of claim 1, wherein the spring mechanism comprises a leaf spring or a torsion spring.

3. The rotational atherectomy device of claim 1, wherein the outer shaft is rotatable.

4. The rotational atherectomy device of claim 3, wherein the rotatable outer shaft is configured to rotate together with the rotatable inner shaft.

5. The rotational atherectomy device of claim 1, wherein the plurality of blades comprises 2 to 10 blades.

6. The rotational atherectomy device of claim 1, wherein at least one of the plurality of blades comprises a cutting edge.

7. The rotational atherectomy device of claim 6, wherein the cutting edge is positioned radially inwardly offset from a radial outermost point of the blade.

8. The rotational atherectomy device of claim 6, wherein the cutting edge is disposed at a circumferential edge of the blade.

9. The rotational atherectomy device of claim 1, wherein at least one of the plurality of blades has a claw-shaped axial cross section.

10. The rotational atherectomy device of claim 1, wherein at least one of the plurality of blades comprises an abrasive surface disposed on a radial outer surface of the blade.

11. (canceled)

12. The rotational atherectomy device of claim 1, wherein the radial outer surface of at least one of the plurality of blades has a curved convex shape.

13. The rotational atherectomy device of claim 1, wherein the plurality of blades are substantially straight along a longitudinal direction.

14. The rotational atherectomy device of claim 1, wherein the outer shaft is in contact with at least one of the plurality of blades.

15. The rotational atherectomy device of claim 1, wherein the outer shaft covers the plurality of blades in the radially contracted configuration and the amount of distal movement of the rotatable inner shaft relative to the outer shaft determines the amount of radial expansion of the blades.

16. (canceled)

17. The rotational atherectomy device of claim 1, wherein the rotatable inner shaft and/or the outer shaft are flexible.

18. (canceled)

19. The rotational atherectomy device of claim 1, further comprising a control unit connected at the proximal end of the rotatable inner shaft and the outer shaft, wherein the control unit comprises a rotational motor for providing rotation to the rotatable inner shaft and is operable to control a speed of the rotational motor.

20. (canceled)

21. (canceled)

22. The rotational atherectomy device of claim 19, wherein the control unit comprises an expansion control mechanism for moving the rotatable inner shaft longitudinally with respect to the outer shaft to thereby move the plurality of blades between the radially contracted configuration and the radially expanded configuration.

23. The rotational atherectomy device of claim 22, wherein the expansion control mechanism comprises a rack and pinion.

24. The rotational atherectomy device of claim 1, wherein the rotatable inner shaft further comprises a guidewire lumen for accommodating a guidewire.

25. A method of cutting or abrading tissue using a rotatable atherectomy device comprising a rotatable inner shaft, a plurality of blades connected to the distal end of the rotatable inner shaft through a spring mechanism which biases the plurality of blades radially outward, and an outer shaft disposed over the rotatable inner shaft; the method comprising:
introducing the atherectomy device into a blood vessel;
advancing the atherectomy device to a position in the blood vessel where tissue is to be cut or abraded;
radially expanding the blades to the diameter of the vessel by distally advancing the inner shaft;
rotating the inner shaft to cut or abrade the tissue.
